(19) United States
(12) Patent Application Publication
(10) Pub. No.: US 2024/0358247 A1
(43) Pub. Date: Oct. 31, 2024

(54) TRAY COUPLING MODULE FOR A CLEANING MACHINE

(71) Applicant: Ecolab USA Inc., Saint Paul, MN (US)

(72) Inventors: Sébastien RUIZ, Saint-Victoret (FR); Vincent BRESSION, Gémenos (FR); Yolande MARTINEZ, La Ciotat (FR); Eric PENDARIES, La Ciotat (FR)

(21) Appl. No.: 18/647,420

(22) Filed: Apr. 26, 2024

Related U.S. Application Data
(60) Provisional application No. 63/499,095, filed on Apr. 28, 2023.

(51) Int. Cl. A61B 1/12

(52) U.S. Cl. CPC A61B 1/123

(57) ABSTRACT

A tray for a cleaning machine is hereby disclosed. In some examples, the tray has a coupling block configured to couple to a module of a cleaning machine while the tray is inserted into the machine. The coupling block can include through-holes configured to fluidly connect at least one medical apparatus lying in the tray to cleaning fluid ports of a cleaning machine. A blind groove and/or C-shaped jaws may be adapted to provide engagement between the coupling block and the cleaning machine.

This application claims priority to U.S. Provisional Application No. 63/499,095, filed on Apr. 28, 2023, the entire contents of which are incorporated herein by reference.

### TECHNICAL FIELD

The present disclosure pertains to the field of cleaning and sterilization systems for healthcare, pharmaceutical or laboratories applications and in particular to the cleaning and sterilization of medical apparatuses such as endoscopes and the like.

### BACKGROUND

Cleaning and sterilization of medical tools is usually performed by machines having a chamber that receives one or more trays where the medical apparatuses to be cleaned are lying. The machine may have fluid sources that project cleaning liquid into the chamber and onto the tray. When the medical apparatus to be cleaned has lumens (e.g., endoscopes, syringes, etc.), it may be necessary to fluidly connect the lumens to the cleaning sources. An operator usually performs this connection manually. This is time consuming and not hygienic. In addition, the risk of error is higher when connecting a lumen to the machine manually inside the chamber. Hence, there is a need to provide a cleaning machine which enables an automatic connection between cleaning sources and the lumens of the medical tools to be cleaned.

### SUMMARY

This disclosure proposes an improved situation, by facilitating the operator's work and by making the process more hygienic.

This disclosure relates to a tray for a cleaning machine comprising: a bottom panel configured to receive at least one medical apparatus; a sidewall extending upwardly from the bottom panel; and a coupling block attached to the sidewall, wherein the coupling block comprises a plurality of through-holes configured to fluidly connect the at least one medical apparatus to cleaning fluid ports of the cleaning machine, and wherein the coupling block is configured to engage a corresponding connection module on a side of a cleaning chamber of the cleaning machine as the tray is inserted into the cleaning machine.

Such a tray enables the operator to connect the medical apparatus to the coupling block in a location remote from the machine. This prevents the need for the operator to manipulate anything in the chamber of the machine. The connection between the cleaning fluid ports and the lumens of the medical apparatus is then performed automatically by the machine: the single insertion motion (i.e., sliding the tray in the machine) enables to align the tray with fluid cleaning ports, without the need for further handling the tray or the medical apparatus.

In another aspect, the coupling block comprises a blind groove adapted to engage an indexing pin of the cleaning machine.

In another aspect, the blind groove comprises a funnel-shaped open-end. The funnel may assist centering the tray with respect to the indexing pin.

In another aspect, the blind groove is parallel to the bottom panel and is located on an outward side surface of the coupling block.

In another aspect, the blind groove has a blind end forming a blind hole that is arranged to enable the indexing pin to penetrate further into the block.

In another aspect, the coupling block has a top surface and a bottom surface, and wherein at least one of the top and bottom surfaces is provided with a reinforcing component configured to engage a C-shaped jaw of the cleaning machine.

In another aspect, the coupling block further comprises at least one permanent magnet configured to be detectable by a sensor of the cleaning machine.

In another aspect, the through-holes are parallel to the bottom panel and are substantially perpendicular to the sidewall.

In another aspect, the at least one medical apparatus is an endoscope having a plurality of lumens, and the plurality of through-holes on the coupling block have an inlet side configured to fluidly couple to corresponding ones of the cleaning fluid ports and an outlet side configured to couple to the plurality of lumens.

The disclosure also relates to a machine for cleaning medical apparatuses, the machine comprising: a chamber adapted to receive a tray and at least one medical apparatus thereon; a module located at a side of the chamber, and configured to couple the machine to the tray, the module comprising: a plurality of cleaning fluid ports protruding into the chamber and adapted to engage and disengage through-holes of a coupling block of the tray; and at least one of a C-shaped jaw and an indexing pin, configured to engage the coupling block as the tray is inserted into the chamber.

In another aspect, the indexing pin is configured to engage a blind groove of the coupling block so as to abut against a blind end of the blind groove when the through-holes align with the cleaning fluid ports.

In another aspect, the module comprises a plate that is movable towards and away from the chamber, and the indexing pin and the cleaning fluid ports are fixed to the plate.

In another aspect, the indexing pin and the cleaning fluid ports are movable by means of an actuator, and wherein the actuator is configured to assume: a first position wherein the indexing pin and the cleaning fluid ports are retracted; a second, intermediate, position, wherein the indexing pin protrudes so as to engage the coupling block of the tray while the cleaning fluid ports do not engage the through-holes of the tray; and a third position wherein the indexing pin and the cleaning fluid ports protrude sufficiently to engage the coupling block of the tray.

In another aspect, the machine further comprises a stopper which is movable between an active position where it prevents the actuator from reaching the third position, and a passive position where the stopper allows the actuator to operate in all three positions.

In another aspect, the C-shaped jaw is configured to engage one of a top and a bottom surface of the coupling block.

In another aspect, the machine further comprises a sensor for detecting the location of a permanent magnet arranged in the coupling block of the tray.

In another aspect, the machine further comprises a first door enabling access to the chamber and a second door, opposite the first door.

In another aspect, the cleaning fluid ports are nozzles.

The disclosure also relates to a system comprising the above-mentioned machine and the above-mentioned tray.

In another aspect, the machine a first door and a second door, opposite the first door, and wherein the indexing pin is configured to abut the blind groove during insertion of the tray through the first door.

The disclosure also relates to a method of operating the above-mentioned system, the method comprising: sliding the tray into the chamber along a first direction until aligning through-holes of the tray with cleaning fluid ports of the cleaning machine; advancing the cleaning fluid ports into the through-holes of the tray; performing the cleaning of the medical apparatus; retracting the cleaning fluid ports; extracting the tray out of the chamber.

In another aspect, during inserting the tray, the indexing pin engages the blind groove of the tray and/or the C-shaped jaw engages the top or bottom surface of the block.

In another aspect, after sliding the tray and prior to advancing the cleaning fluid ports, a stopper is automatically retracted upon detecting that the tray is located such that the through-holes align with the cleaning fluid ports.

In another aspect, the tray comprises permanent magnets arranged in the block and detecting the position of the tray comprises detecting the position of the permanent magnets by means of sensors provided in the module.

In another aspect, the machine comprises a first door and a second door, opposite the first door, and wherein inserting the tray is performed through the first door and extracting the tray is performed through the second door.

### BRIEF DESCRIPTION OF DRAWINGS

Other features, details and advantages will be shown in the following detailed description and on the figures, on which:

FIG. 1 is an example of a tray according to an embodiment.

FIG. 2 is an example of a module for coupling the tray of FIG. 1 to a cleaning machine.

FIGS. 3 to 6 show various configurations of the module in use.

FIG. 7 is a diagram of an exemplary method.

### DETAILED DESCRIPTION

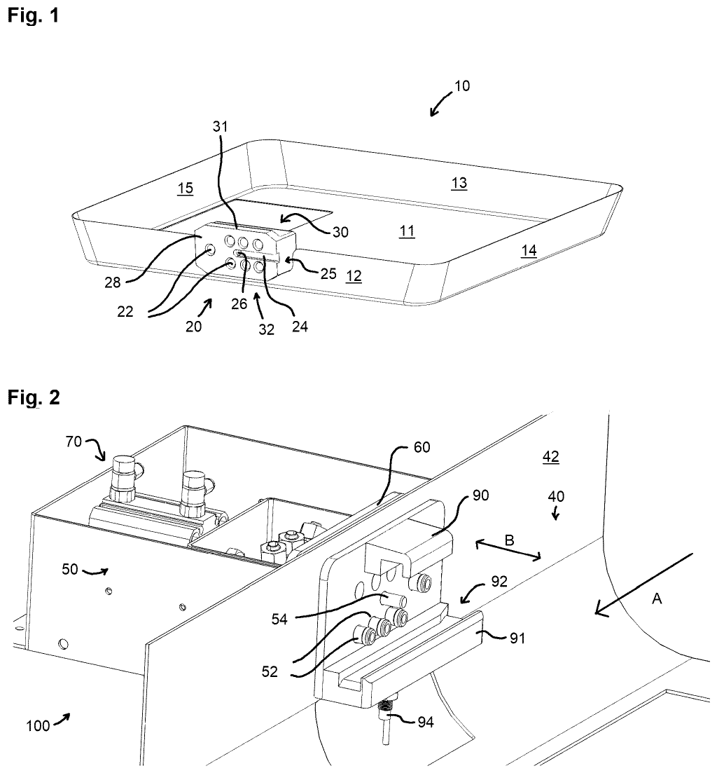

FIG. 1 shows an example of a tray 10 according to the present disclosure. The tray 10 has a bottom panel 11 and may have four sidewalls 11, 12, 13, 14. The tray 10 may be made of any suitable material and may be of any suitable size. As visible on FIG. 1, the tray 10 may have an opening arranged on the bottom panel 11 to let cleaning fluid flow down during a cleaning process. A medical apparatus to be cleaned, such as an endoscope, may be positioned on the bottom panel 11. The tray 10 is intended to be positioned in a cleaning machine, so that the medical apparatus located thereon may be cleaned.

A sidewall 12 of the tray may be provided with a coupling block 20. This block is intended to fluidly connect inner cavities of the medical apparatus to cleaning fluid ports (i.e. cleaning nozzles, cleaning sources) of the machine. To that end, the block 20 contains through-holes 22. One end of the through-holes 22 (the end that is visible on FIG. 1) can be connected to cleaning fluid ports of the cleaning machine while the opposite ends of the through-holes (not visible on FIG. 1) are intended to be connected to the medical apparatus laying on the bottom panel 11.

In the example shown, there are seven through-holes 22 but any other number of through-holes may be chosen. Also, the through-holes 22 appear here as having the same diameter. The person skilled in the art would recognize that it may be advantageous to provide a higher or lower number of through-holes and/or to provide through-holes of various diameters, depending on the medical apparatus(es) that are to be cleaned.

The block 20 may be provided with a blind groove 24. The groove 24 is blind in the sense that it has an open end 25 that is open on a front surface of the block 20, and an opposite blind end 26 that does not open at one side of the block. The blind end 26 forms a blind hole in a direction that is parallel to the through-holes 22 for advantageous reasons that will be explained below.

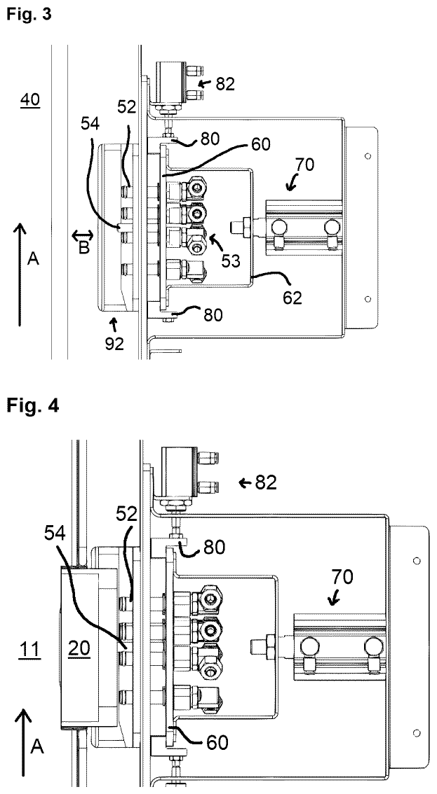

The blind groove 24 is intended to cooperate with an indexing pin (see number 54 on FIG. 2) of the cleaning machine when the tray is inserted into the machine. When the tray 10 is moved in a direction from left to right on the FIG. 1, the indexing pin of the cleaning machine, which extends perpendicularly to the side surface 28, engages the groove 24, first through the open end 25 and then slides until it abuts with the blind end 26. To facilitate the penetration of the indexing pin in the groove, the open end 25 may be shaped as a funnel.

In the example shown, the groove 24 extends in a direction that is parallel to the bottom panel 11 and that is essentially horizontal in use. Other orientations may be possible for the groove 24.

The through-holes 22 are shown as being transversal to the groove 24 but they may have another orientation. Also, the through-holes 22 are shown to be linear but they may have a more complex design. In an alternative embodiment, the through-holes may be arranged vertically, or they may have an inlet on a side (to be connected to the cleaning fluid port) that has a different orientation than the outlet on an opposite side of the block (that is to be connected to the medical apparatus).

Alternatively, or in complement with the groove 24, the block 20 may have a top surface 30 and a bottom surface 32. One or both of these surfaces 30, 32 may be provided with a reinforcing portion 31. As will be shown below, the top and bottom surfaces 30, 32 may cooperate with jaws of the machine such as to guide the sliding motion of the tray during its insertion. These jaws may also hold the block 20 and the tray 10 immobile despite the force resulting from the high-pressure fluid that is injected through the through-holes 22.

In the block 20, and in particular in the vicinity of the top or bottom surface 30, 32, one or more permanent magnets may be inserted, so as to assist the cleaning machine to detect the location of the tray 10. Any other technology for detecting the presence, the location or the identification of the tray may be used alternatively or in conjunction with the magnets (e.g., RFID, QR-code, optical or physical pattern, etc.).

Although the tray 10 shown on FIG. 1 comprises one block 20 and one groove 24, the tray 10 is not limited to this design and can comprise more than one groove 24 on a block to cooperate with respective indexing pins of the machine. The tray may also contain more than one coupling block 20 in order to connect additional medical apparatuses to additional cleaning fluid ports of the machine. In that case, not all the coupling blocks need to be provided with a groove 24.

FIG. 2 shows (in part) a cleaning machine 100 for cleaning medical apparatuses. The machine 100 has a chamber 40 that is partially shown. The tray of FIG. 1 is intended to enter the chamber 40 along the direction indicated with the arrow A. The machine 100 may have a single door allowing access of the tray to the chamber 40 or may have two doors. In that latter case, the tray will exit the chamber 40 along the direction A as well.

On a side 42 of the chamber 40, the machine 100 is provided with a module 50 intended to couple the machine 100 to the tray 10.

The module 50 comprises cleaning fluid ports 52 which protrude in the chamber 40. The cleaning fluid ports 52 may be in the shape of nozzles or may have a different appropriately chosen form. The ports 52 are movable in a direction B that is transverse (and may be perpendicular) to the direction A. The cleaning ports 52 are intended to penetrate in the through-holes 22 of the tray 10, only when the tray is in an appropriate position in the chamber 40. At the distal end of the cleaning ports 52 annular seals may be provided to tightly connect the ports 52 to the through-holes 22. The directions A and B may define, together, a horizontal plane.

The module 50 also comprises an indexing pin 54 protruding in the chamber 40. The indexing pin 54 is movable in the direction B and is intended to engage the blind groove 24 of the tray and to stop the insertion movement of the tray by abutting to the blind end 26 of the groove 24.

In the example of FIG. 2, the ports 52 and the pin 54 are fixed to a plate 60. This plate 60 is moved by means of a linear actuator 70 (e.g., pneumatic or hydraulic piston).

In an alternative embodiment, the pin 54 is movable along a direction that is distinct from the direction of movement of the ports and/or the ports 52 and the pin 54 are moved by independent actuators.

The top and bottom surfaces 30, 32 of the tray 10 can engage C-shaped jaws 90, 91 provided in the module 50. These jaws 90, 91 comprise a groove that cooperates with reinforcement 31 of the tray 10. These jaws 90, 91 assist the positioning of the tray during insertion. For instance, a funnel-shaped end 92 can be foreseen in one or both of the jaws 90, 91. During use, the jaws are also helpful to withstand the force resulting from the injection of fluid through the ports 52 and through the through-holes 22.

The module 50 may also comprise one or more sensors 94 to detect the position of the tray 10, and especially to confirm the precise position of the tray 10 before moving the ports 52 towards the tray 10. In an example, the sensor 94 is a Hall-effect sensor aiming at detecting permanent magnets embedded into the block 20 of the tray 10. In the example shown, the sensor 94 is arranged at the C-shaped jaw but other positions may be considered.

As a consequence, both the coupling block 20 and the module 50 of the machine gather several functions in a compact location: guiding the tray, indexing the tray in position, fluidly connecting the machine to the medical apparatus, holding the tray and counteracting the fluid pressure force, and detecting the position of the tray.

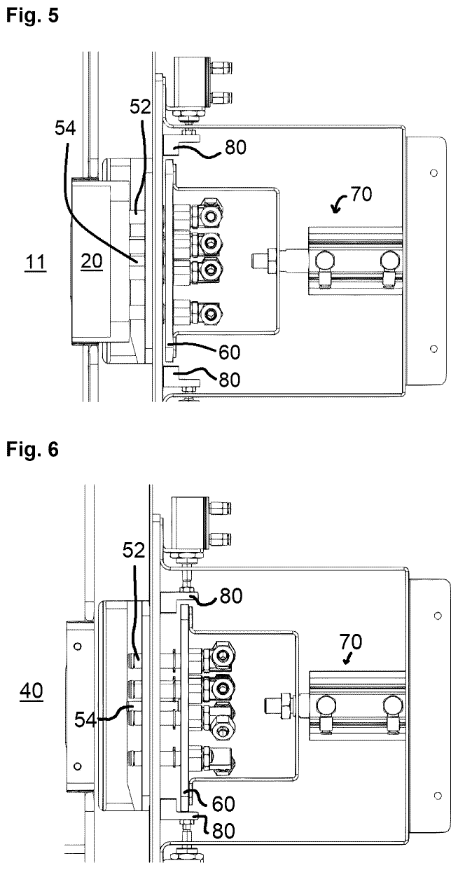

FIGS. 3 to 6 show the various positions of the ports 52 and pin 54, in a top view. To ease the understanding of these figures, the top C-shaped jaw 90 is not represented on FIGS. 3 to 6.

In FIG. 3, the module 50 is in an intermediate position, which is the position that is foreseen to receive and index the tray in position: the indexing pin 54 protrudes in the path of the groove 24 while the ports 52 are not protruding entirely, so as to not interfere with the block 20 of the tray.

This position is guaranteed by the deployment of stoppers 80 which prevent the movement of the plate 60 and the movement of the actuator 70 further towards the chamber 40.

An additional actuator 82 may handle the position of the stoppers 80 (a second actuator is not shown at the bottom of FIG. 3).

FIG. 3 also shows that the plate 60 holding the ports 52 and the pin 54 can be connected to the actuator 70 by means of a U-shape flange 62. This flange 62 delimits a space that can accommodate connectors 53 making possible for the ports 52 to be fed with cleaning media.

FIG. 4 shows the module in the same configuration, once the tray 10 has been inserted in the chamber 40. The groove 24 (visible on FIG. 1) has engaged the indexing pin 54 and the movement of the tray 10 in the direction A has been stopped due to the contact between the indexing pin 54 and the blind end 26 of the groove 24. In that configuration, there is still a clearance between the ports 52 and the block 20.

Upon detecting that the tray has reached the position of FIG. 4, for example with the sensor(s) 94 detecting one or more permanent magnet embedded in the block 20, the machine may automatically control the actuator 82 to retract the stoppers 80 out of the path of the plate 60.

FIG. 5 shows the stoppers 80 once they are retracted off the plate 60 and thereby allow the actuator 70 to push the plate 60 further towards the chamber 40, so as to engage the ports 52 into the through holes 22. This movement is also made possible due to the blind hole 26 which enables the indexing pin 54 that is held by the same plate 60 as the ports 52 to be pushed further into the block 20.

This position is held during injection of cleaning fluid through the cleaning ports 52. The cleaning process may contain various additional steps that may be carried out independently from the injection of fluid through the ports 52.

During cleaning, the C-shaped jaws 90, 91 hold the tray 10 and in particular compensate the force resulting from the injection of fluid which tends to push the tray away from the ports 52.

Once all the necessary cleaning steps have been performed, the tray 10 may be extracted from the chamber. To release the tray 10, the actuator 70 is retracted beyond the position shown on FIG. 3, so that not only the ports 52 but also the indexing pin 54 are cleared off the block 20. After the retractation of the plate 60, the stoppers 80 may be expanded again.

This final position is shown on FIG. 6, once the tray has left the area.

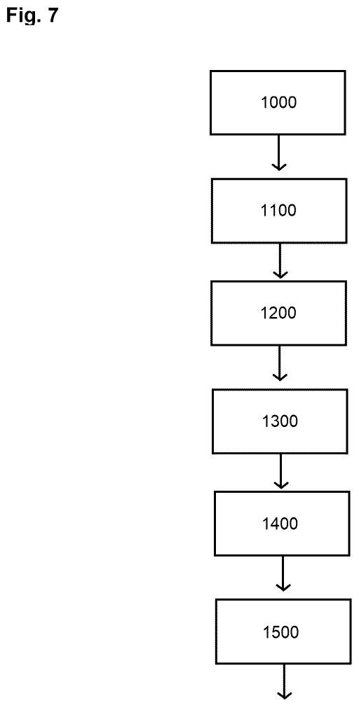

FIG. 7 shows a diagram of the successive operations. In step 1000, the module 50 is in its configuration of FIG. 3 and the tray may be inserted into the chamber 40. The tray 10 may be inserted by hand by an operator. Prior to the insertion of the tray, the operator may have connected medical apparatuses such as endoscopes or the like to the ends of the through-holes 22 that are located on the inner side of the tray 10 (not visible on FIG. 1). The operator may then only hold the tray at one end thereof, without his hand entering the chamber 40.

The tray 10 is inserted into the chamber 40 by sliding along direction A. By this single motion, the coupling block 20 connects to the module 50.

A precise centering and positioning of the tray is helpful to properly align the through-holes 22 with the ports 52. This precise centering and positioning is achieved by the indexing pin 54 which engages the groove 24 and/or by one or both C-shaped jaws 90, 91 which engage the top or bottom surface 30, 32 of the block 20. The tray 10 is slid into the chamber until the through-holes 22 align with the ports 52 (while the ports are in their retracted configuration). This alignment happens when the indexing pin 54 abuts against the blind end 26 of the groove 24. Alternatively, if the tray is void of groove 24, the tray 10 is inserted until another retractable abutment stops its movement at this location.

Once the tray is in a position where the ports 52 align with the through-holes 22, the tray remain immobile until it is extracted out of the chamber.

In step 1100, upon detection of the final position of the tray 10, a control unit of the machine may automatically order the actuators 82 to retract the stoppers 80. This releases the plate 60.

In step 1200, the ports 52 advance to penetrate in the through-hole 22 and establish a fluid connection between a source of cleaning fluid and the lumens of the medical apparatus lying in the tray. In the exemplary embodiment discussed above, this means that the actuator 70 is activated to push the plate 60 forward in the direction of the chamber 40. Since the tray 10 is in such a position that the through-holes 22 align with the ports 52, the actuation of the actuator 70 leads the ports 52 to penetrate into the through-holes 22. The indexing pin 54 which is already in contact with the block 20 will move further into the block 20 in the blind hole 26.

In that position, the cleaning process 1300 is performed.

Once cleaning is done, the ports 52 and the indexing pin 54 are retracted 1400, by moving the plate 60 away from the chamber 40 under the actuation of the actuator 70.

In that position, the tray may be extracted 1500 from the chamber. If the machine is a pass-through cleaning machine, the tray 10 may continue in the same direction A. If the machine is a one-door machine, the tray 10 may be extracted in a direction opposite to arrow A.

Although the preferred embodiments referred above show an indexing pin 54 guiding and stopping the tray at a specific location, another abutment may be foreseen at another location of the chamber and may cooperate with the block 20 or with another portion of the tray 10. This alternative abutment can be retractable or not. For instance, if the machine has a single door, the abutment may not need to be retractable as there is no need to let the tray pass through.

The method described, as well as the tray and the machine discussed above, enable to provide an efficient and more hygienic cleaning process.

As will be apparent to the person skilled in the art, many changes may be made to the present disclosure without departing from the spirit and scope of the disclosure. Also, any feature of any particular embodiment discussed above may be combined with any other feature of any other embodiment.

1. A tray for a cleaning machine, the tray comprising:
a bottom panel configured to receive at least one medical apparatus;
a sidewall extending upwardly from the bottom panel; and
a coupling block attached to the sidewall,
wherein the coupling block comprises a plurality of through-holes configured to fluidly connect the at least one medical apparatus to cleaning fluid ports of the cleaning machine, and
wherein the coupling block is configured to engage a corresponding connection module on a side of a cleaning chamber of the cleaning machine as the tray is inserted into the cleaning machine.

2. The tray of claim 1, wherein the coupling block comprises a blind groove adapted to engage an indexing pin of the cleaning machine.

3. The tray of claim 2, wherein the blind groove is parallel to the bottom panel and is located on an outward side surface of the coupling block.

4. The tray of claim 2, wherein the blind groove has a blind end forming a blind hole that is arranged to enable the indexing pin to penetrate further into the block.

5. The tray of claim 1, wherein the coupling block has a top surface and a bottom surface, and wherein at least one of the top and bottom surfaces is provided with a reinforcing component configured to engage a C-shaped jaw of the cleaning machine.

6. The tray of claim 1, wherein the coupling block further comprises at least one permanent magnet configured to be detectable by a sensor of the cleaning machine.

7. The tray of claim 1, wherein the through-holes are parallel to the bottom panel and are substantially perpendicular to the sidewall.

8. The tray of claim 1, wherein the at least one medical apparatus is an endoscope having a plurality of lumens, and the plurality of through-holes on the coupling block have an inlet side configured to fluidly couple to corresponding ones of the cleaning fluid ports and an outlet side configured to couple to the plurality of lumens.

9. A machine for cleaning medical apparatuses, the machine comprising:
a chamber adapted to receive a tray and at least one medical apparatus thereon;
a module located at a side of the chamber, and configured to couple the cleaning machine to the tray, the module comprising:
a plurality of cleaning fluid ports protruding into the chamber and adapted to engage and disengage through-holes of a coupling block of the tray; and
at least one connection feature configured to engage the coupling block as the tray is inserted into the chamber.

10. The machine of claim 9, wherein the at least one connection feature is a C-shaped jaw and/or an indexing pin.

11. The machine of claim 10, wherein the indexing pin is configured to engage a blind groove of the coupling block so as to abut against a blind end of the blind groove when the through-holes align with the cleaning fluid ports.

12. The machine of claim 10, wherein the module comprises a plate that is movable towards and away from the chamber, and the indexing pin and the cleaning fluid ports are fixed to the plate.

13. The machine of claim 10, wherein the indexing pin and the cleaning fluid ports are movable by means of an actuator, and wherein the actuator is configured to assume:
a first position wherein the indexing pin and the cleaning fluid ports are retracted;
a second, intermediate, position, wherein the indexing pin protrudes so as to engage the coupling block of the tray while the cleaning fluid ports do not engage the through-holes of the tray; and
a third position wherein the indexing pin and the cleaning fluid ports protrude sufficiently to engage the coupling block of the tray.

14. The machine of claim 13, further comprising a stopper which is movable between an active position where it prevents the actuator from reaching the third position, and a passive position where the stopper allows the actuator to operate in all three positions.

15. The machine of claim 10, wherein the C-shaped jaw is configured to engage one of a top and a bottom surface of the coupling block.

16. The machine of claim 9, further comprising a sensor for detecting the location of a permanent magnet arranged in the coupling block of the tray.

17. The machine of claim 9, wherein the cleaning fluid ports are nozzles.

18. A method of operating a cleaning machine, the method comprising:
sliding a tray containing at least one medical apparatus into a chamber of a cleaning machine along a first direction until through-holes of the tray that are fluidly connected to fluid ports of the at least one medical apparatus are aligned with cleaning fluid ports of the cleaning machine;
advancing cleaning fluid ports into the through-holes of the tray;
performing a cleaning of the medical apparatus by at least delivering fluid through the cleaning fluid ports and into the fluid ports of the at least one medical apparatus;
retracting the cleaning fluid ports; and
extracting the tray out of the chamber.

19. The method of claim 18, wherein sliding the tray comprises causing an indexing pin to engage a blind groove of the tray and/or a C-shaped jaw to engage top and/or bottom surfaces of a coupling block of the tray.

20. The method of claim 18, wherein after sliding the tray and prior to advancing the cleaning fluid ports, a stopper is automatically retracted upon detecting that the tray is located such that the through-holes align with the cleaning fluid ports.

21. The method of claim 18, wherein the cleaning machine is an endoscope reprocessing machine and the at least medical apparatus is an endoscope.
